(19) United States
(12) Patent Application Publication
(10) Pub. No.: US 2018/0296192 A1
(43) Pub. Date: Oct. 18, 2018

(54) ULTRASOUND DIAGNOSIS APPARATUS

(71) Applicant: Konica Minolta, Inc., Tokyo (JP)

(72) Inventor: Tomohito SAKAI, Yokohama-shi (JP)

(21) Appl. No.: 15/947,337

(22) Filed: Apr. 6, 2018

(30) Foreign Application Priority Data
Apr. 18, 2017 (JP) 2017-081732

(51) Int. Cl. A61B 8/08; A61B 8/00

(52) U.S. Cl. CPC A61B 8/5207; A61B 8/4444; A61B 8/5246; A61B 8/54

(57) ABSTRACT

An ultrasound diagnosis apparatus includes: a transmitter that outputs a drive signal to an ultrasound probe that transmits and receives ultrasound waves; a receiver that obtains a reception signal from the ultrasound probe; an image generator that generates ultrasound image data from the reception signal; a multiple-image-saved image data acquisition part that obtains multiple-image-saved image data containing a plurality of ultrasound images generated in the past within a single screen; an information acquisition part that obtains an image arrangement format of the plurality of ultrasound images of the obtained multiple-image-saved image data; and a display controller that performs comparison display of a comparison image among the plurality of ultrasound images of the multiple-image-saved image data and a current image based on the generated current ultrasound image data onto a single screen of the display part in accordance with the obtained image arrangement format.

The entire disclosure of Japanese patent Application No. 2017-081732, filed on Apr. 18, 2017, is incorporated herein by reference in its entirety.

### BACKGROUND

### Technological Field

The present invention relates to an ultrasound diagnosis apparatus.

### Description of the Related Art

Ultrasound diagnosis is capable of obtaining an ultrasound image of heart beats and fetal movement by simple operation of applying an ultrasound probe from the body surface while ensuring safety, and thus, can be used for repetitive examination. There is a known ultrasound diagnosis apparatus used for performing ultrasound diagnosis including generating and displaying an ultrasound image.

The field of orthopedic ultrasound often includes a user scene related to paired sites such as hands, in which a target site (affected portion side, or affected side) as a painful site (chief complaint) and its opposite site (healthy portion side, or healthy side) are compared with each other in two-image parallel display. While whether to obtain an image from the affected portion side or obtain the image from the healthy portion side is determined on the case-by-case basis depending on conditions and circumstances of the patient, an ultimate image would be an image displayed as a frozen image with a fixed layout in which the healthy portion side is displayed on the left or upper side and the affected portion side is displayed on the right or lower side, or vice versa. In many cases, the image is applied after determining the order of arrangement of the healthy portion side and the affected portion side, and thus, the left-side site of the body is not necessarily displayed in the image on the left side. In addition, a parallel display screen of the two images, that is, the images of the healthy portion side and the affected portion side can be saved as multiple-image (or two-image)-saved image data as a single piece of image data.

There is also known a medical image diagnosis apparatus that displays a recorded image (saved image) and a diagnostic real-time image (live image) in parallel in a manner so as to be able to change the size and display position of the image in accordance with an operator's input (refer to JP 2005-288043 A). The recorded image can be displayed in a split display.

Unfortunately, however, the conventional ultrasound diagnosis apparatus including the above-described conventional medical image diagnosis apparatus has difficulty in properly displaying the two-image-saved image data as a comparison image. FIG. 12A is a diagram illustrating a two-image-saved image 700 in a left-right image arrangement format. FIG. 12B is a diagram illustrating a two-image comparison screen 800 in the left-right image arrangement format. FIG. 13A is a diagram illustrating a two-image-saved image 900 in the upper-lower image arrangement format. FIG. 13B is a diagram illustrating a two-image comparison screen 1000 in the left-right image arrangement format. Note that the figures including FIGS. 12A to 13B and other ultrasound images omit illustrations of displayed content of ultrasound images.

For example, it is assumed that the two-image-saved image data containing the two-image-saved image 700 having the left-right image arrangement format illustrated in FIG. 12A is saved in a storage of a conventional ultrasound diagnosis apparatus. The image arrangement format is a parallel arrangement format of two ultrasound images contained in one piece of two-image-saved image data, either the left-right format or upper-lower format. The two-image-saved image 700 includes a past ultrasound image 710 arranged on the left side and a past ultrasound image 720 arranged on the right side. The ultrasound image 710 is, for example, an ultrasound image of the healthy portion side of a predetermined patient scanned in the past. The ultrasound image 720 is, for example, an ultrasound image of the affected portion side of the same patient as the predetermined patient scanned in the past.

Here is an exemplary case of performing comparison display of the two-image-saved image 700 saved in the past and a current frozen image, on a conventional ultrasound diagnosis apparatus. In this case, the conventional ultrasound diagnosis apparatus reads two-image-saved image data of the two-image-saved image 700 from the storage to display the two-image comparison screen 800 illustrated in FIG. 12B. The two-image comparison screen 800 includes a past ultrasound image 830 arranged on the left side and an ultrasound image 840 as the current frozen image arranged on the right side. The ultrasound image 830 corresponds to an ultrasound image region 730 of the two-image-saved image 700, and includes a right-side portion of the ultrasound image 710 and a left-side portion of the ultrasound image 720. This is because there is no information of which ultrasound image of the two-image-saved image 700 is to be displayed as the comparison image, and thus, the ultrasound image region 730 at a center portion of the two-image-saved image 700 is adopted as the comparison image. The ultrasound image 840 is an ultrasound image on the current affected portion side of the same patient scanned on the two-image-saved image 700, for example.

In comparison of the ultrasound images of an affected portion side between the past image and the current image of a predetermined patient, the two-image comparison screen 800 includes an irrelevant ultrasound image of the past on the healthy portion side, resulting in an occurrence of a difference in the display range of the ultrasound images of the affected portion side between the past image and the current image. This makes it difficult to perform proper comparison.

Moreover, it is assumed, for example, that the two-image-saved image data containing the two-image-saved image 900 in the upper-lower image arrangement format illustrated in FIG. 13A is stored in a storage of a conventional ultrasound diagnosis apparatus. The two-image-saved image 900 includes a past ultrasound image 910 arranged on the upper side and a past ultrasound image 920 arranged on the lower side. The ultrasound image 910 is an ultrasound image of the healthy portion side of a predetermined patient scanned in the past, for example. The ultrasound image 920 is an ultrasound image on the affected portion side of the same patient in the predetermined patient scanned in the past, for example.

Here is an exemplary case where the conventional ultrasound diagnosis apparatus reads two-image-saved image data of the two-image-saved image 900 from the storage so as to display the two-image comparison screen 1000 illustrated in FIG. 13B. The two-image comparison screen 1000 includes an ultrasound image 1030 of the past arranged on the left side and an ultrasound image 1040 of the current frozen image arranged on the right side. The ultrasound image 1030 corresponds to an ultrasound image region 930 of the two-image-saved image 900, and includes a lower-side portion of the ultrasound image 910 and an upper-side portion of the ultrasound image 920. This is because there is no information of which ultrasound image of the two-image-saved image 1000 is to be displayed as the comparison image, and thus, the ultrasound image region 930 at a center portion of the two-image-saved image 1000 is adopted as the comparison image. Moreover, there is no information related to an image arrangement format of the two-image-saved image 900, and thus, the two-image comparison screen 1000 displays the ultrasound images 1030 and 1040 in the left-right image arrangement format. The ultrasound image 1040 is an ultrasound image on the current affected portion side of the same patient scanned on the two-image-saved image 900, for example.

In comparison of the ultrasound images of an affected portion side between the past image and the current image of a predetermined patient, the two-image comparison screen 1000 includes an irrelevant ultrasound image of the past on the healthy portion side, resulting in an occurrence of a difference in the size of the ultrasound images of the affected portion side between the past image and the current image. In addition, the image arrangement format (upper-lower format) of the two-image-saved image 900 differs from the image arrangement format (left-right format preset by the conventional ultrasound diagnosis apparatus) of the two-image comparison screen 1000. This hinders displaying an appropriate region of the ultrasound image on the affected portion side of the past, making it difficult to perform proper comparison.

### SUMMARY

An object of the present invention is to appropriately perform comparison display of a multiple-image-saved image and a current ultrasound image.

To achieve the abovementioned object, according to an aspect of the present invention, an ultrasound diagnosis apparatus reflecting one aspect of the present invention comprises:

a transmitter that outputs a drive signal to an ultrasound probe that transmits and receives ultrasound waves;

a receiver that obtains a reception signal from the ultrasound probe;

an image generator that generates ultrasound image data from the reception signal;

a multiple-image-saved image data acquisition part that obtains multiple-image-saved image data containing a plurality of ultrasound images generated in the past within a single screen;

an information acquisition part that obtains an image arrangement format of the plurality of ultrasound images of the obtained multiple-image-saved image data; and

a display controller that performs comparison display of a comparison image among the plurality of ultrasound images of the multiple-image-saved image data and a current image based on the generated current ultrasound image data onto a single screen of the display part in accordance with the obtained image arrangement format.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an external view of an ultrasound diagnosis apparatus according to an embodiment of the present invention;

FIG. 2 is a block diagram illustrating a functional configuration of an ultrasound diagnosis apparatus;

FIG. 3 is a flowchart illustrating first multiple-image-saved image saving processing;

FIG. 4 is a diagram illustrating a first two-image-saved image in a left-right image arrangement format;

FIG. 5 is a diagram illustrating a first two-image-saved image in an upper-lower image arrangement format;

FIG. 6 is a flowchart illustrating first multiple-image comparison display processing;

FIG. 7 is a diagram illustrating a first two-image comparison screen in a left-right image arrangement format;

FIG. 8 is a diagram illustrating a first two-image comparison screen in an upper-lower image arrangement format;

FIG. 9 is a flowchart illustrating second multiple-image-saved image saving processing;

FIG. 10 is a flowchart illustrating second multiple-image comparison display processing;

FIG. 11 is a flowchart illustrating third multiple-image comparison display processing;

FIG. 12A is a diagram illustrating a second two-image-saved image in the left-right image arrangement format;

FIG. 12B is a diagram illustrating a second two-image comparison screen in the left-right image arrangement format;

FIG. 13A is a diagram illustrating a second two-image-saved image in the upper-lower image arrangement format; and

FIG. 13B is a diagram illustrating a third two-image comparison screen in the left-right image arrangement format.

### DETAILED DESCRIPTION OF EMBODIMENTS

Hereinafter, one or more embodiments and first to third modifications according to the present invention will be sequentially described in detail with reference to the drawings. However, the scope of the invention is not limited to the disclosed embodiments.

### Embodiments

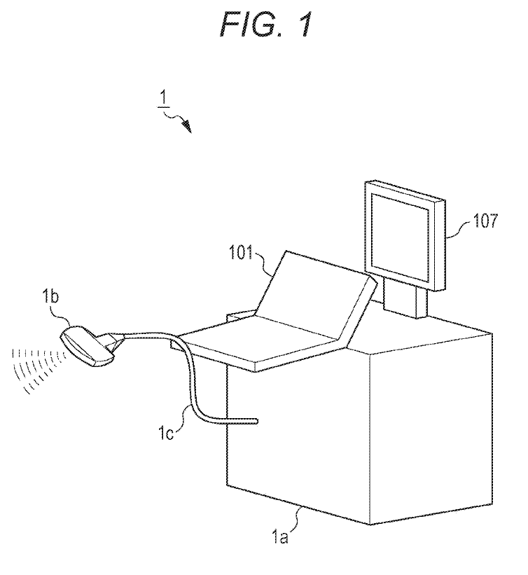

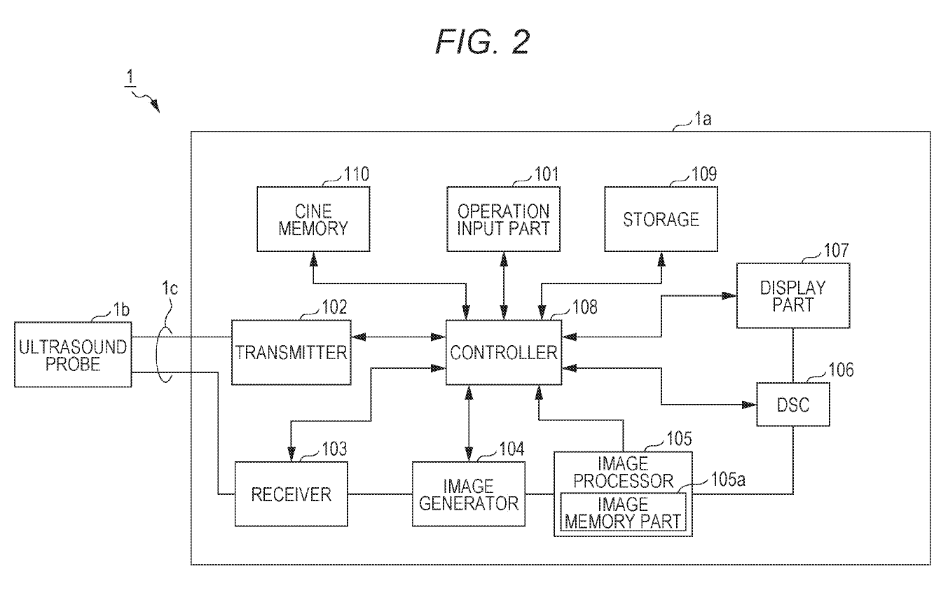

The present embodiment will be described with reference to FIGS. 1 to 8. First, an apparatus configuration according to the present embodiment will be described with reference to FIGS. 1 and 2. FIG. 1 is an external view of an ultrasound diagnosis apparatus 1 according to the present embodiment. FIG. 2 is a block diagram illustrating a functional configuration of the ultrasound diagnosis apparatus 1.

As illustrated in FIGS. 1 and 2, the ultrasound diagnosis apparatus 1 according to the present embodiment includes an ultrasound diagnosis apparatus main body 1a and an ultrasound probe 1b. The ultrasound probe 1b transmits ultrasound waves (transmission ultrasound waves) to a subject such as a living body (not illustrated), and receives reflected waves of the ultrasound waves (reflected ultrasound waves) reflected by the subject. The ultrasound diagnosis apparatus main body 1a is connected to the ultrasound probe 1b via a cable 1c and transmits a drive signal of an electric signal to the ultrasound probe 1b to allow the ultrasound probe 1b to transmit a transmission ultrasound to the subject. Together with this, the ultrasound diagnosis apparatus main body 1a forms an image as an ultrasound image of an internal state of the subject on the basis of a reception signal being an electric signal generated by the ultrasound probe 1b in accordance with the reflected ultrasound waves from the inside of the subject received by the ultrasound probe 1b.

The ultrasound probe 1b includes transducers formed with piezoelectric elements. The plurality of the transducers is arranged in a one-dimensional array in the azimuth direction, for example. The present embodiment uses the ultrasound probe 1b including 192 transducers, for example. Note that the transducers may be arranged in a two-dimensional array. Moreover, the number of transducers can be set to any number. Moreover, while the present embodiment employs a linear scanning electronic scanning probe as the ultrasound probe 1b, either the electronic scanning probe or a mechanical scanning probe may be employed. It is possible to employ any of the linear scanning probe, a sector scanning probe, and a convex scanning probe so as to replace the ultrasound probe 1b with another type.

As illustrated in FIG. 2, the ultrasound diagnosis apparatus main body 1a includes: an operation input part 101; a transmitter 102; a receiver 103; an image generator 104; an image processor 105; a digital scan converter (DSC) 106; a display part 107; a controller 108, a storage 109 serving as a multiple-image-saved image data acquisition part, an information acquisition part, and a display controller; and a cine memory 110.

The operation input part 101 includes various switches, buttons, a trackball, a mouse, and a keyboard, for performing input of data such as a command to instruct a diagnosis start from an operator (including a doctor and a technician), and personal information of the subject, for example, and outputs an operation signal to the controller 108. Moreover, the operation input part 101 may have a configuration that includes a touch screen provided on the display screen of the display part 107.

The transmitter 102 is a circuit that supplies, under the control of the controller 108, a drive signal being an electric signal to the ultrasound probe 1b via the cable 1c to cause the ultrasound probe 1b to generate a transmission ultrasound wave. Moreover, the transmitter 102 includes a clock generation circuit, a delay circuit, and a pulse generation circuit, for example. The clock generation circuit is a circuit that generates a clock signal that determines a transmission timing and a transmission frequency of the drive signal. The delay circuit is a circuit for setting a delay time for each of individual paths for which transmission timings of the drive signals for each of the transducers are set, and delaying the transmission of the drive signal by the set delay time to achieve convergence of the transmission beam formed by the transmission ultrasound waves. The pulse generation circuit is a circuit for generating a pulse signal as a drive signal at a predetermined cycle. The above-configured transmitter 102 drives a continuous portion (for example, 64) among a plurality (for example, 192) of transducers arranged in the ultrasound probe 1b to generate transmission ultrasound waves. In this, the transmitter 102 performs scanning by azimuthally shifting the transducer to be driven every time the transmission ultrasound wave is generated.

The receiver 103 is a circuit to receive a reception signal being an electric signal from the ultrasound probe 1b via the cable 1c under the control of the controller 108. The receiver 103 includes an amplifier, an A/D conversion circuit, and a phasing-addition circuit, for example. The amplifier is a circuit to amplify the reception signal with a preset amplification factor for each of individual paths corresponding to each of the transducers. The A/D conversion circuit is a circuit for A/D converting the amplified reception signal. The phasing-addition circuit gives a delay time to the A/D converted reception signal for each of the individual paths corresponding to each of the transducers so as to adjust a time phase and perform addition of these phases (phasing-addition) to generate sound ray data.

The image generator 104 performs envelope detection processing, log compression, or the like, on the sound ray data from the receiver 103, and then applies dynamic range adjustment and gain adjustment to the data to perform luminance conversion so as to generate B-mode image data. That is, the B-mode image data represents the intensity of the reception signal by luminance. The image generator 104 may be configured to be capable of generating A-mode image data, M-mode image data, and image data by the Doppler method in addition to B-mode image data.

The image processor 105 includes an image memory part 105a formed of a semiconductor memory such as a dynamic random access memory (DRAM). The image processor 105 stores the B-mode image data output from the image generator 104 in the image memory part 105a on a frame basis. Image data defined on a frame basis may be referred to as ultrasound image data or frame image data in some cases. The frame image data stored in the image memory part 105a is transmitted to the DSC 106 under the control of the controller 108.

The DSC 106 converts the frame image data received from the image processor 105 into an image signal for the display part 107 and outputs the signal to the display part 107.

The display part 107 may be formed by applying a display device such as a liquid crystal display (LCD), a cathode-ray tube (CRT) display, an organic electronic luminescence (EL) display, an inorganic EL display, and a plasma display. The display part 107 displays an image on the display screen in accordance with the image signal output from the DSC 106.

The controller 108 includes a central processing unit (CPU), a read only memory (ROM), and a random access memory (RAM), for example, reads various processing programs such as a system program stored in the ROM, expands the programs onto the RAM, and centrally controls the operation of individual components of the ultrasound diagnosis apparatus 1 in accordance with the developed program. The ROM is constituted with a non-volatile memory such as a semiconductor and stores a system program, various processing programs executable on the system program, and various data such as a gamma table, corresponding to the ultrasound diagnosis apparatus 1. Example of the various programs include a first multiple-image-saved image saving program for executing first multiple-image-saved image saving processing to be described below and a first multiple-image comparison display program for executing first multiple-image comparison display processing. These programs are stored in the form of computer-readable program codes, and the CPU sequentially executes operation corresponding to the program codes. The RAM forms a work area for temporarily storing various programs to be executed by the CPU and data related to these programs.

The storage 109 is constituted with a large-capacity recording medium such as a hard disk drive (HDD), for example, and stores ultrasound image data, two-image-saved image data described below, various types of setting information, or the like, to be saved in association with patient information.

The cine memory 110 is a storage constituted with a RAM or the like, performing first-in-first-out (FIFO) storage of a predetermined number of frames, and storing, as cine image data, image data of a plurality of frames of a live moving image updated in real time, under the control of the controller 108. Alternatively, the cine memory 110 may be constituted by a portion of the RAM of the controller 108.

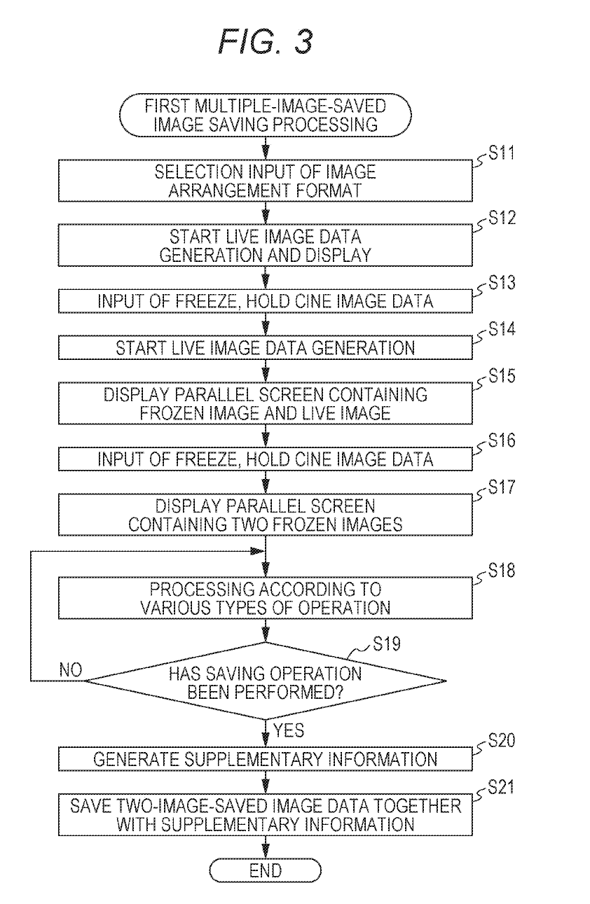

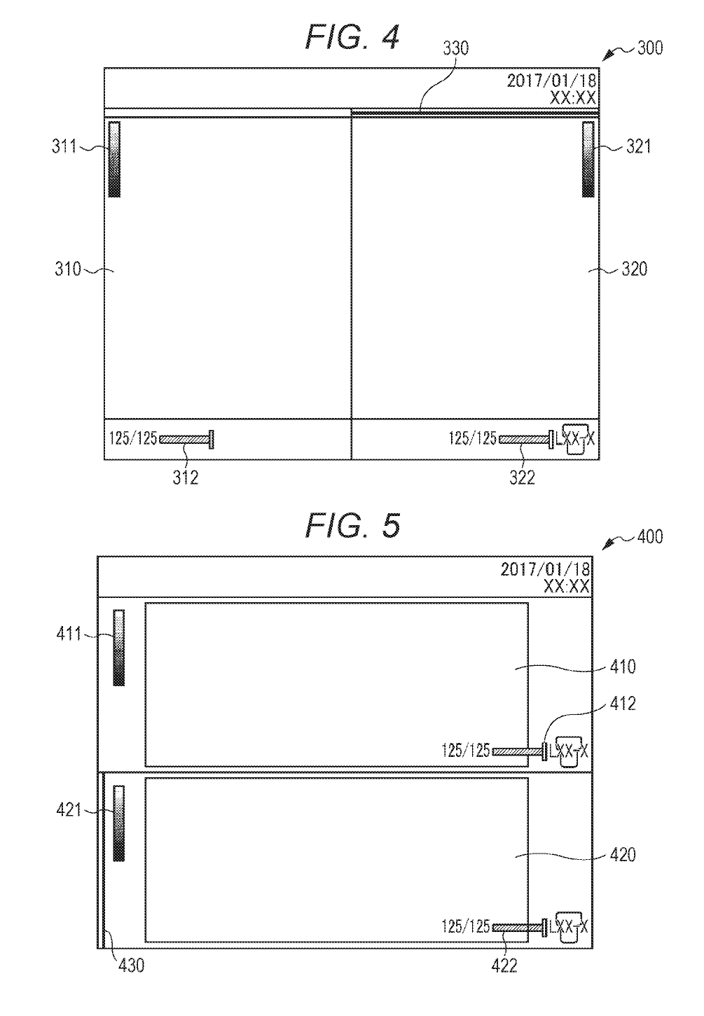

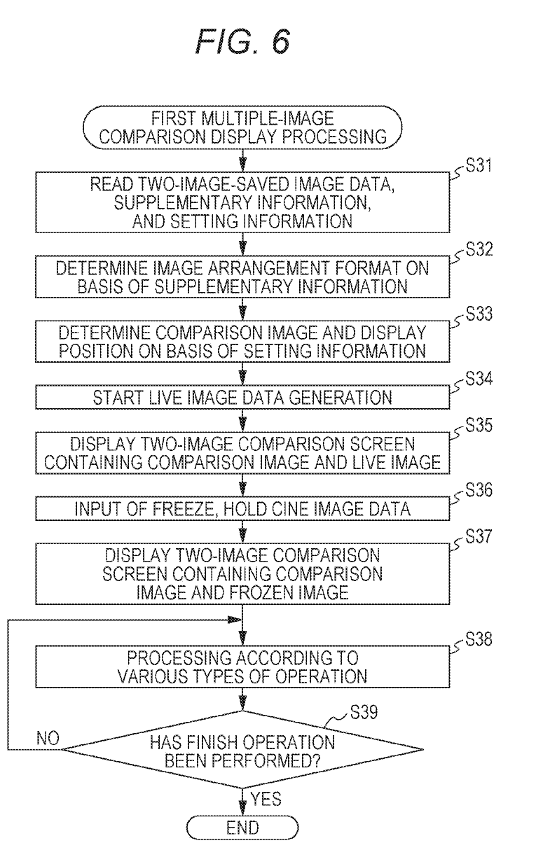

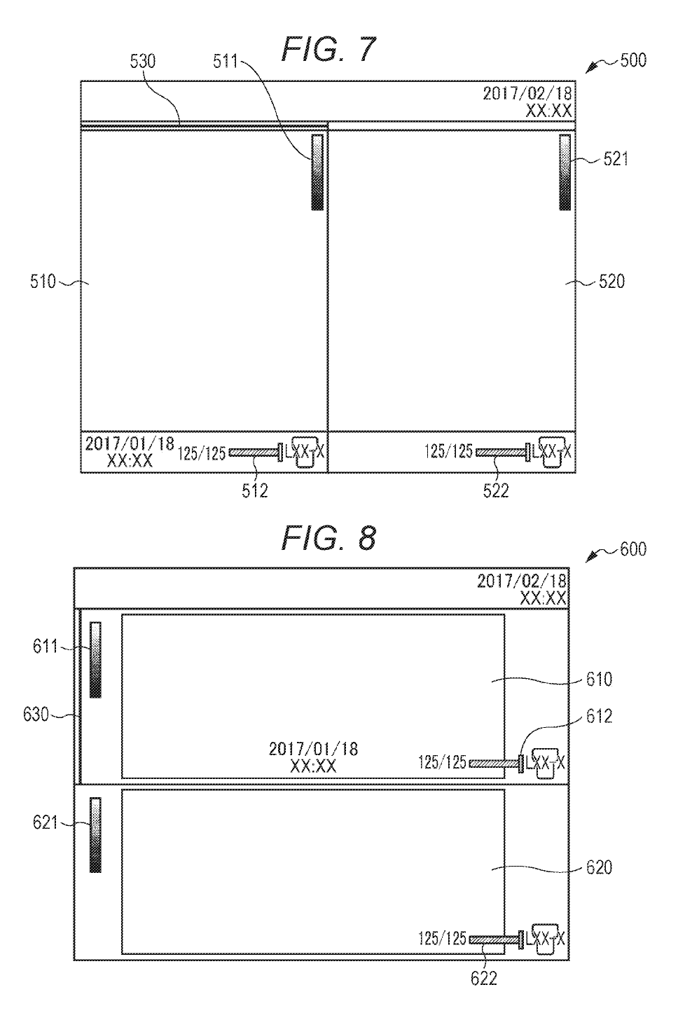

Next, operation of the ultrasound diagnosis apparatus 1 will be described with reference to FIGS. 3 to 8. FIG. 3 is a flowchart illustrating the first multiple-image-saved image saving processing. FIG. 4 is a diagram illustrating a two-image-saved image 300 in a left-right image arrangement format. FIG. 5 is a diagram illustrating a two-image-saved image 400 in an upper-lower image arrangement format. FIG. 6 is a flowchart illustrating the first multiple-image comparison display processing. FIG. 7 is a diagram illustrating a two-image comparison screen 500 in the left-right image arrangement format. FIG. 8 is a diagram illustrating a two-image comparison screen 600 in the upper-lower image arrangement format.

First, setting information preset and stored in the storage 109 will be described. In the present embodiment, the setting information is to include comparison image designation information and comparison image display position information. The comparison image designation information indicates which of left-right (or upper-lower) ultrasound images among two-image-saved image data which is one piece of image data containing two ultrasound images scanned in the past is to be designated as a comparison image. The comparison image display position information indicates in which of the left-right (or the upper-lower) positions comparison images are to be arranged and displayed on the two-image comparison screen for comparing the two ultrasound images.

The comparison image designation information is information to designate, for example, “right” as a comparison image in a case where the image arrangement format of the two-image-saved image data is the left-right format and designate “lower” as the comparison image in a case where the image arrangement format is the upper-lower format. The comparison image display position information indicates that the comparison image be arranged in “left” in a case where the image arrangement format of the two-image-saved image data is left-right, and in a case where the image arrangement format is the same, the comparison image is to be arranged in “upper” in a case where the image arrangement format is the upper-lower format.

In advance, in the ultrasound diagnosis apparatus 1, the controller 108 receives an operator's operation input of the setting information of the display position information via the operation input part 101, generates the setting information including the input comparison image display position information, and stores the generated information in the storage 109. Moreover, it is assumed that the comparison image designation information of the setting information is to be fixedly set in the ultrasound diagnosis apparatus 1. Note that the comparison image display position information of the setting information may also be fixedly set in the ultrasound diagnosis apparatus 1.

Next, the first multiple-image-saved image saving processing will be described with reference to FIGS. 3 to 5. The first multiple-image-saved image saving processing is processing of transmitting and receiving ultrasound waves to and from the subject to generate two ultrasound images and saving the two-image-saved image data containing the two generated ultrasound images arranged in parallel. In the ultrasound diagnosis apparatus 1, an operator's input of an instruction to execute the first multiple-image-saved image saving processing via the operation input part 101 triggers the controller 108 to execute the first multiple-image-saved image saving processing in accordance with the first multiple-image-saved image saving program stored in the ROM.

As illustrated in FIG. 3, the controller 108 first receives an operator's input of an image arrangement format for display and storage of two ultrasound images to be scanned via the operation input part 101 (step S11). The image arrangement format is either the left-right format or the upper-lower format.

Subsequently, in accordance with the operator's input of scan start instruction, or the like, via the operation input part 101, the controller 108 controls the transmitter 102, the receiver 103, the image generator 104, the image processor 105, the DSC 106, the display part 107, and the cine memory 110 to start generating and displaying live image data on the display part 107, while storing cine image data of the live image into the cine memory 110 (step S12). The live image is a real-time ultrasound image (B-mode image).

The operator applies the ultrasound probe 1b to a target site of the subject and moves the probe as appropriate, and prepares for freeze input while visually confirming the live image. Step S12 aims to generate an ultrasound image of the site on the healthy portion side, for example. Subsequently, the controller 108 receives an operator's operation input of freeze via the operation input part 101, and holds the cine image data of a predetermined number of frames corresponding to the input operation of freeze, onto the cine memory 110 (step S13).

Subsequently, the controller 108 starts generation (step S14) of live image data similarly to step S12. Subsequently, the controller 108 follows the image arrangement format input in step S11 to generate a parallel screen containing a frozen image based on the cine image data generated in step S13 and a live image of the live image data being arranged in parallel, and displays the generated screen on the display part 107 (step S15). Note that arrangement on the parallel screen of step S15 is assumed to be preset such that the frozen image is arranged on the left side and the live image is arranged on the right side, for example. Accordingly, a frozen image being an ultrasound image on the healthy portion side is arranged on the left side.

The operator applies the ultrasound probe 1b to the target site of the subject and moves the probe as appropriate, and prepares for freeze input while visually confirming the live image. Step S15 aims to generate an ultrasound image of a site on the affected portion side, for example. Subsequently, the controller 108 receives an operator's operation input of freeze via the operation input part 101, and holds the cine image data of a predetermined number of frames corresponding to the input operation of freeze, onto the cine memory 110 (step S16).

Subsequently, the controller 108 follows the image arrangement format input in step S11 to generate a parallel screen containing a frozen image based on the cine image data generated in step S13 and a frozen image based on the cine image data generated in step S16 being arranged in parallel, and starts displaying the generated screen on the display part 107 (step S17). Note that arrangement on the parallel screen of step S17 is assumed to be preset such that the frozen image in step S13 is arranged on the left side and the frozen image in step S16 is arranged on the right side, for example. Accordingly, a frozen image being an ultrasound image on the healthy portion side is arranged on the left side, and a frozen image being an ultrasound image on the affected portion side is arranged on the right side.

In step S17, a parallel screen similar to the two-image-saved image 300 illustrated in FIG. 4 is displayed, for example. Herein, it is assumed that the image arrangement format is the left-right format. The two-image-saved image 300 includes an ultrasound image 310 arranged on the left side, an ultrasound image 320 arranged on the right side, and further includes grayscale bars 311 and 321, cine bars 312 and 322, and an active screen line 330, as graphic information.

The ultrasound image 310 is an ultrasound image on the healthy portion side, for example. The ultrasound image 320 is an ultrasound image on the affected portion side, for example. The grayscale bars 311 and 321 are scale (chart) images of a black and white gradation. The grayscale bar 311 is arranged on the left side portion of the ultrasound image 310. The grayscale bar 321 is arranged on the right side portion of the ultrasound image 320.

The cine bars 312 and 322 includes a bar main body and a knob. The bar main body extends in the left-right direction, and a plurality of frames of cine image data is associated with a position in the left-right direction. The knob is an operation element operable to move in the left-right direction on the bar main body. The cine bar 312 is arranged below the ultrasound image 310. The cine bar 322 is arranged below the ultrasound image 320. The knob is operated to move on the cine bar 312 to select cine image data corresponding to the position of the knob from among the cine image data generated in step S13, and the ultrasound image corresponding to the selected cine image data is displayed on the ultrasound image 310. The knob is operated to move on the cine bar 322 to select cine image data corresponding to the position of the knob from among the cine image data generated in step S16, and the ultrasound image corresponding to the selected cine image data is displayed on the ultrasound image 320.

The active screen line 330 is a display element indicating an active (selected) state of the ultrasound image, and switches between an active state of the ultrasound image 310 and an active state of the ultrasound image 320 in accordance with active switching operation. The active screen line 330 is arranged above the ultrasound image in the active state. It is possible to apply processing according to the operation corresponding to the ultrasound image on the ultrasound image in the active state. For example, it is possible to perform processing of selecting cine image data according to the operation of the cine bar corresponding to the ultrasound image in the active state.

Subsequently, the controller 108 receives operator's various operation inputs via the operation input part 101, and performs processing in accordance with the operation content (step S18). Examples of the operation and processing in step S18 include the switching operation of the active state, the left-right (ultrasound image) display switching on the active screen line, the cine bar knob moving operation corresponding to the ultrasound image in the active state, and cine image data selection (change of ultrasound image to display) corresponding to the knob movement.

Subsequently, the controller 108 receives operator's saving operation input for the two-image-saved image data via the operation input part 101, and determines whether the saving operation has been performed (step S19). In a case where the saving operation has not been performed (step S19; NO), the processing returns to step S18. In a case where the saving operation has been performed (step S19; YES), the controller 108 generates supplementary information including the image arrangement format input in step S11 (step S20).

Subsequently, the controller 108 generates a parallel screen being displayed on the display part 107 as two-image-saved image data, stores the generated two-image-saved image data in the storage 109 (step S21) in association with the supplementary information generated in step S20, and finishes the first multiple-image-saved image saving processing. Step S21 saves the two-image-saved image data of the two-image-saved image 300, for example. The two-image-saved image data and the supplementary information are stored in association with patient information input by the operator via the operation input part 101.

While the above description of the first multiple-image-saved image saving processing is an exemplary case where the image arrangement format is the left-right format, the image arrangement format may be the upper-lower format. In this case, step S17 displays a parallel screen similar to the two-image-saved image 400 illustrated in FIG. 5. The two-image-saved image 400 includes an ultrasound image 410 arranged on the upper side, an ultrasound image 420 arranged on the lower side, and further includes grayscale bars 411 and 421, cine bars 412 and 422, and an active screen line 430, as graphic information.

The ultrasound image 410 is an ultrasound image on the healthy portion side, for example. The ultrasound image 420 is an ultrasound image on the affected portion side, for example. The grayscale bar 411 is arranged on the left side of the ultrasound image 410. The grayscale bar 421 is arranged on the left side of the ultrasound image 420.

The cine bar 412 is arranged in the lower right side portion of the ultrasound image 410. The cine bar 422 is arranged at the lower right side portion of the ultrasound image 420. The knob is operated to move on the cine bar 412 to select cine image data corresponding to the position of the knob from among the cine image data generated in step S13, and the ultrasound image corresponding to the selected cine image data is displayed on the ultrasound image 410. The knob is operated to move on the cine bar 422 to select cine image data corresponding to the position of the knob from among the cine image data generated in step S16, and the ultrasound image corresponding to the selected cine image data is displayed on the ultrasound image 420.

The active screen line 430 is arranged on the left side of the ultrasound image in the active state in accordance with the active switching operation. Step S20 saves the two-image-saved image data of the two-image-saved image 400, for example.

Next, the first multiple-image comparison display processing will be described with reference to FIGS. 6 to 8. The first multiple-image comparison display processing is processing of using the supplementary information and the setting information to perform comparison display of an appropriate ultrasound image contained in the two-image-saved image data stored in the storage 109 and a current frozen image, being arranged in parallel.

It is assumed that the first multiple-image-saved image saving processing is executed at least once beforehand and at least one two-image-saved image is stored in the storage 109 beforehand. In the ultrasound diagnosis apparatus 1, an operator's input of an instruction to execute the first multiple-image comparison display processing via the operation input part 101 triggers the controller 108 to execute the first multiple-image comparison display processing in accordance with the first multiple-image comparison display program stored in the ROM.

As illustrated in FIG. 6, the controller 108 receives an operator's selection input of two-image-saved image data via the operation input part 101, and reads the selected two-image-saved image data and associated supplementary information and setting information from the storage 109 (step S31). In step S31, for example, two-image-saved image data associated with patient information of a subject (patient) to be diagnosed and scanned in the past is selected.

Subsequently, the controller 108 determines (step S32) the image arrangement format on the basis of the supplementary information read in step S31. For example, the left-right format is determined as the image arrangement format.

Subsequently, the controller 108 determines an ultrasound image as a comparison image among the two-image-saved image data on the basis of the comparison image designation information of the setting information read in step S31, and determines the display position of the comparison image on a two-image comparison screen described below on the basis of the comparison image display position information of the setting information in a same manner (step S33). For example, the ultrasound image on the right side among the two-image-saved image data is determined as the comparison image, and the left side is determined as the comparison image display position on the two-image comparison screen.

Subsequently, the controller 108 starts generation of live image data similarly to step S14 of the first multiple-image-saved image saving processing (step S34). Step S34 is a step to generate a live image of a site on the affected portion side, for example.

Subsequently, the controller 108 follows the image arrangement format and the comparison image display position determined in steps S32 and S33 to generate a two-image comparison screen containing the comparison image determined in step S33 among the two-image-saved image data read in step S31 and a live image being arranged in parallel, and displays the generated screen on the display part 107 (step S35). For example, in the two-image comparison screen in step S35, the comparison image is arranged on the left side and the live image is arranged on the right side. Accordingly, a comparison image being a past ultrasound image on the affected portion side of the subject is arranged on the left side, and a live image being a current ultrasound image on the affected portion side of the subject is arranged on the right side.

The operator applies the ultrasound probe 1b to the target site of the subject and moves the probe as appropriate, and prepares for freeze input while visually confirming the live image. Step S35 aims to generate an ultrasound image of a site on the affected portion side, for example. Subsequently, the controller 108 receives an operator's operation input of freeze via the operation input part 101, and holds the cine image data of a predetermined number of frames corresponding to the input of freeze, onto the cine memory 110 (step S36).

Subsequently, the controller 108 follows the image arrangement format and the comparison image display position determined in steps S32 and S33 to generate a two-image comparison screen containing the comparison image determined in step S33 among the two-image-saved image data read in step S31 and a frozen image based on the cine image data generated in step S35 being arranged in parallel, and starts displaying the generated screen on the display part 107 (step S37). In the two-image comparison screen in step S37, for example, the comparison image of step S31 is arranged on the left side and the frozen image of step S36 is arranged on the right side. Accordingly, a comparison image being a past ultrasound image on the affected portion side of the subject is arranged on the left side, and a frozen image being a current ultrasound image on the affected portion side of the subject is arranged on the right side.

In step S37, for example, the two-image comparison screen 500 illustrated in FIG. 7 is displayed. Herein, it is assumed that the image arrangement format is the left-right format. The two-image comparison screen 500 includes an ultrasound image 510 arranged on the left side, an ultrasound image 520 arranged on the right side, grayscale bars 511 and 521, cine bars 512 and 522, and an active screen line 530.

The ultrasound image 510 is a past ultrasound image on the affected portion side, for example, and has the same content as the ultrasound image 320 of the two-image-saved image 300. The ultrasound image 520 is a current ultrasound image of the affected portion side, for example. The grayscale bar 511 is arranged on the right side portion of the ultrasound image 510 and has the same content as the grayscale bar 321. The grayscale bar 521 is arranged on the right side portion of the ultrasound image 520.

The cine bar 512 is arranged below the ultrasound image 510 and has the same content as the cine bar 322 and is inoperable since this is a portion of the image. The cine bar 522 is arranged below the ultrasound image 520 and is operable. The knob is operated to move on the cine bar 522 to select cine image data corresponding to the position of the knob from among the cine image data generated in step S36, and the ultrasound image corresponding to the selected cine image data is displayed on the ultrasound image 520.

The active screen line 530 is arranged on the ultrasound image 510 being in the active state at the time of saving and has the same content as the active screen line 330 and is inoperable since this is a portion of the image.

Subsequently, the controller 108 receives operator's various operation inputs via the operation input part 101, and performs processing according to the operation content (step S38). Examples of the operation and processing in step S37 include cine bar knob moving operation corresponding to a live image and cine image data selection (changing ultrasound image to be displayed) according to the knob movement.

Subsequently, the controller 108 receives operator's operation input of finishing the first multiple-image comparison display processing via the operation input part 101, and determines whether the finish operation has been performed (step S39). In a case where the finish operation has not been performed (step S39; NO), the processing returns to step S38. In a case where the finish operation is performed (step S39; YES), the first multiple-image comparison display processing is finished.

While the above description of the first multiple-image comparison display processing is an exemplary case where the image arrangement format of the two-image-saved image data (supplementary information) is the left-right format, the image arrangement format of the two-image-saved image data may be the upper-lower format. In this case, step S37 displays the two-image comparison screen 600 illustrated in FIG. 8, for example. The two-image comparison screen 600 includes an ultrasound image 610 arranged on the upper side, an ultrasound image 620 arranged on the lower side, grayscale bars 611 and 621, cine bars 612 and 622, and an active screen line 630.

The ultrasound image 610 is, for example, a past ultrasound image on the affected portion side and has the same content as the ultrasound image 420 of the two-image-saved image 400. The ultrasound image 620 is a current ultrasound image on the affected portion side, for example. The grayscale bar 611 is arranged on the left side of the ultrasound image 610 and has the same content as the grayscale bar 421. The grayscale bar 621 is arranged on the left side of the ultrasound image 620.

The cine bar 612 is arranged at the lower right portion side of the ultrasound image 610 and has the same content as the cine bar 422 and is inoperable since this is a portion of the image. The cine bar 622 is arranged at the lower right portion of the ultrasound image 620 and is operable. The knob is operated to move on the cine bar 622 to select cine image data corresponding to the position of the knob from among the cine image data generated in step S36, and the ultrasound image corresponding to the selected cine image data is displayed on the ultrasound image 620.

The active screen line 630 is arranged on the left side of the ultrasound image 610 being in the active state at the time of saving and has the same content as the active screen line 430 and is inoperable since this is a portion of the image.

According to the present embodiment as described above, the ultrasound diagnosis apparatus 1 includes the transmitter 102 that outputs a drive signal to the ultrasound probe 1b that transmits and receives ultrasound waves, the receiver 103 that obtains a reception signal from the ultrasound probe 1b, and the image generator 104 that generates ultrasound image data from the reception signal. The ultrasound diagnosis apparatus 1 further includes the controller 108 that reads and obtains two-image-saved image data having a plurality of ultrasound images generated in the past in a single screen, from the storage 109, then obtains the image arrangement format of the plurality of ultrasound images of the obtained two-image-saved image data, and performs comparison display of the comparison image among the plurality of ultrasound images of the two-image-saved image data and a current frozen image as a current image based on the generated current ultrasound image data, onto a single screen of the display part 107 in accordance with the obtain image arrangement format.

With this configuration, it is possible to perform comparison display of the past comparison image of the two-image-saved image data and the current frozen image in an appropriate image arrangement format in accordance with the obtained image arrangement format, with the same display range for the comparison image and the current frozen image, leading to achievement of operator's visual comparison of the two ultrasound images with higher accuracy.

Moreover, the controller 108 obtains the image arrangement format from the supplementary information stored in the storage 109 in association with the obtained two-image-saved image data, that is, the supplementary information having the image arrangement format of the two-image-saved image data. This makes it possible to appropriately perform comparison display of the past comparison image of the two-image-saved image data and the current frozen image, in the image arrangement format of the two-image-saved image data.

In addition, the controller 108 obtains comparison image designation information for designating a comparison image from among the plurality of ultrasound images of the obtained two-image-saved image data, and performs comparison display of the past comparison image of the two-image-saved image data and the current frozen image on the basis of the obtained image arrangement format and comparison image designation information. This makes it possible to perform comparison display of an appropriate past comparison image among the two-image-saved image data, and the current frozen image.

Moreover, the controller 108 reads from the storage 109 the setting information containing comparison image designation information fixedly set beforehand, and obtains comparison image designation information from the setting information. This makes it possible to perform comparison display of a fixedly set appropriate past comparison image among the two-image-saved image data, and the current frozen image.

(First Modification)

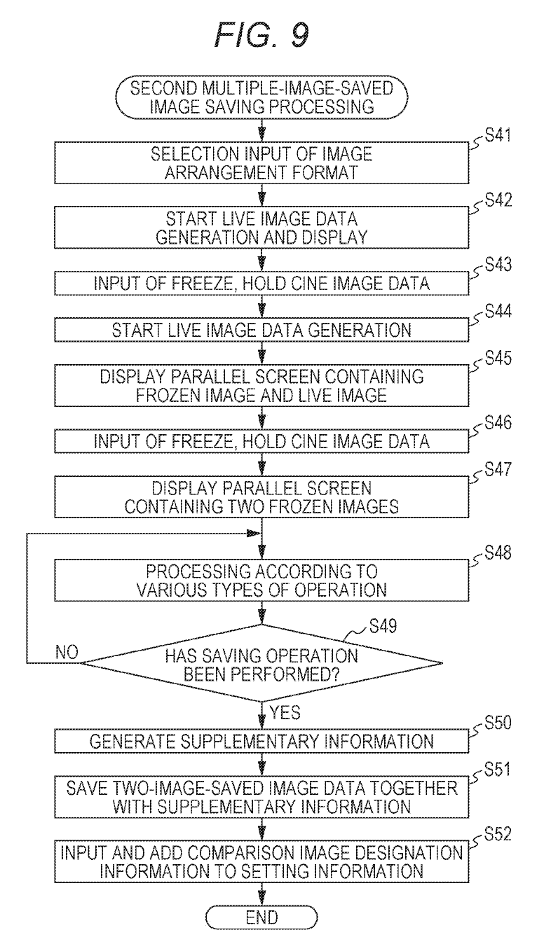

A first modification of the above embodiment will be described with reference to FIG. 9. FIG. 9 is a flowchart illustrating second multiple-image-saved image saving processing.

In the present modification, the ultrasound diagnosis apparatus 1 is used as an apparatus, similarly to the above-described embodiment. It is assumed, however, that the ROM of the controller 108 stores a second multiple-image-saved image saving program instead of the first multiple-image-saved image saving program. In addition, it is assumed that the setting information set and stored beforehand in the storage 109 includes the comparison image display position information alone.

Next, operation of the ultrasound diagnosis apparatus 1 according to the present modification will be described. The second multiple-image-saved image saving processing will be described with reference to FIG. 9. The second multiple-image-saved image saving processing is processing of transmitting and receiving ultrasound waves to and from the subject to generate two ultrasound images, saving the two-image-saved image data containing the two generated ultrasound images arranged in parallel, and adding comparison image designation information to the setting information in accordance with an operator's input.

In the ultrasound diagnosis apparatus 1, an operator's input of an instruction to execute the second multiple-image-saved image saving processing via the operation input part 101 triggers the controller 108 to execute the second multiple-image-saved image saving processing in accordance with the second multiple-image-saved image saving program stored in the ROM.

As illustrated in FIG. 9, steps S41 to S51 are respectively similar to steps S11 to S21 of the first multiple-image-saved image saving processing in FIG. 3. Subsequently, the controller 108 receives an operator's selection input of an ultrasound image to be the comparison image from among the two-image-saved image data via the operation input part 101, adds the comparison image designation information corresponding to the input selection information to the setting information stored in the storage 109 (step S52), and finishes the second multiple-image-saved image saving processing. In a case where the image arrangement format is the left-right format, for example, the ultrasound image on the right side among the two-image-saved image data is selected as the comparison image. In a case where the image arrangement format is the upper-lower format, for example, the ultrasound image on the lower side among the two-image-saved image data is selected as the comparison image.

It is allowable to use a configuration in which step S52 is executed before the step S51, or the first multiple-image-saved image saving processing is finished after the execution of the step S51 and step S52 is executed as appropriate before the execution of the first multiple-image comparison display processing. Moreover, similarly to the above-described embodiment, the first multiple-image-saved image display processing is executed after execution of the second multiple-image-saved image saving processing.

According to the present modification as described above, the controller 108 reads the setting information containing the comparison image designation information set beforehand in accordance with an operator's operation input from the storage 109, and obtains the comparison image designation information from the setting information. This makes it possible to perform comparison display of an appropriate past comparison image according to an operator's intention among the two-image-saved image data, and the current frozen image.

(Second Modification)

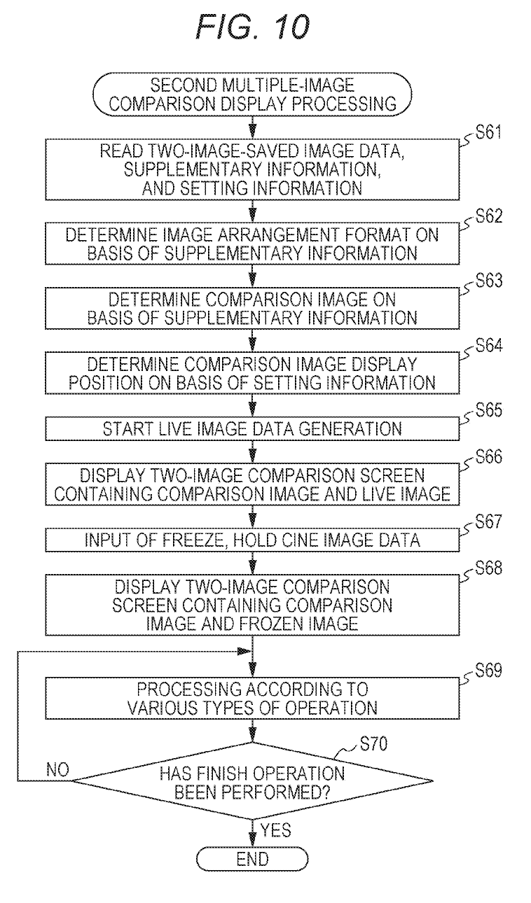

A second modification of the above embodiment will be described with reference to FIG. 10. FIG. 10 is a flowchart illustrating second multiple-image comparison display processing.

In the present modification, the ultrasound diagnosis apparatus 1 is used as an apparatus, similarly to the above-described embodiment. It is assumed, however, that the ROM of the controller 108 stores a second multiple-image comparison display program instead of the first multiple-image comparison display program. In addition, it is assumed that the setting information set and stored beforehand in the storage 109 includes the comparison image display position information alone.

Next, operation of the ultrasound diagnosis apparatus 1 according to the present modification will be described. The second multiple-image comparison display processing will be described with reference to FIG. 10. The second multiple-image comparison display processing is processing of using the supplementary information and the setting information to perform comparison display of an appropriate ultrasound image contained in the two-image-saved image data stored in the storage 109 and a current frozen image, being arranged in parallel.

It is assumed that the first multiple-image-saved image saving processing is executed at least once beforehand and at least one two-image-saved image is stored in the storage 109 beforehand. In step S20 of the first multiple-image-saved image saving processing, however, the controller 108 generates supplementary information including the image arrangement format input in step S11 and comparison image designation information of designating the ultrasound image switched to the active state in step S18 as the comparison images.

In the ultrasound diagnosis apparatus 1, an operator's input of an instruction to execute the second multiple-image comparison display processing via the operation input part 101 triggers the controller 108 to execute the second multiple-image comparison display processing in accordance with the second multiple-image comparison display program stored in the ROM.

As illustrated in FIG. 10, steps S61 and S62 are respectively similar to steps S31 and S32 of the first multiple-image comparison display processing in FIG. 6. Subsequently, the controller 108 determines (step S63) an ultrasound image as a comparison image among the two-image-saved image data on the basis of the comparison image designation information of the supplementary information read in step S61. For example, in a case where the image arrangement format is the left-right format, the ultrasound image on the right side among the two-image-saved image data is determined as the comparison image. In a case where the image arrangement format is the upper-lower format, for example, the ultrasound image on the lower side among the two-image-saved image data is determined as the comparison image.

Subsequently, the controller 108 determines (step S64) the comparison image display position on the two-image comparison screen described below on the basis of the comparison image display position information of the setting information read in step S61. In a case where the image arrangement format is the left-right format, for example, the left side is determined as the comparison image display position on the two-image comparison screen. In another case where the image arrangement format is the upper-lower format, for example, the upper side is determined as the comparison image display position on the two-image comparison screen.

Steps S65 to S70 are respectively similar to steps S34 to S39 of the first multiple-image comparison display processing in FIG. 6.

As described above, according to the present modification, the controller 108 obtains the comparison image designation information from the supplementary information associated with the obtained two-image-saved image data, that is, the supplementary information containing the comparison image designation information designating the ultrasound image that is in the active state at the time of saving of the two-image-saved image data, as the comparison image. This makes it possible to perform comparison display of an appropriate past comparison image that is in the active state at the time of saving among the two-image-saved image data, and the current frozen image.

(Third Modification)

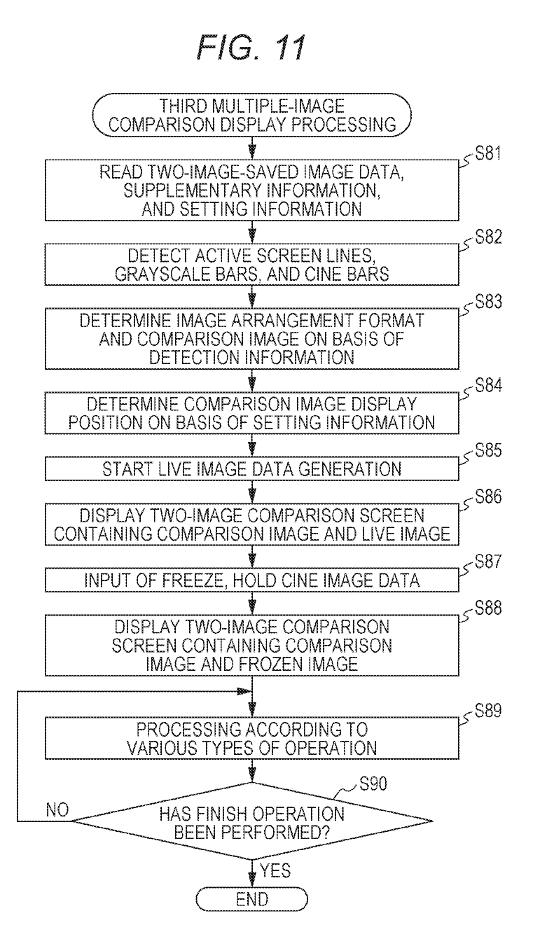

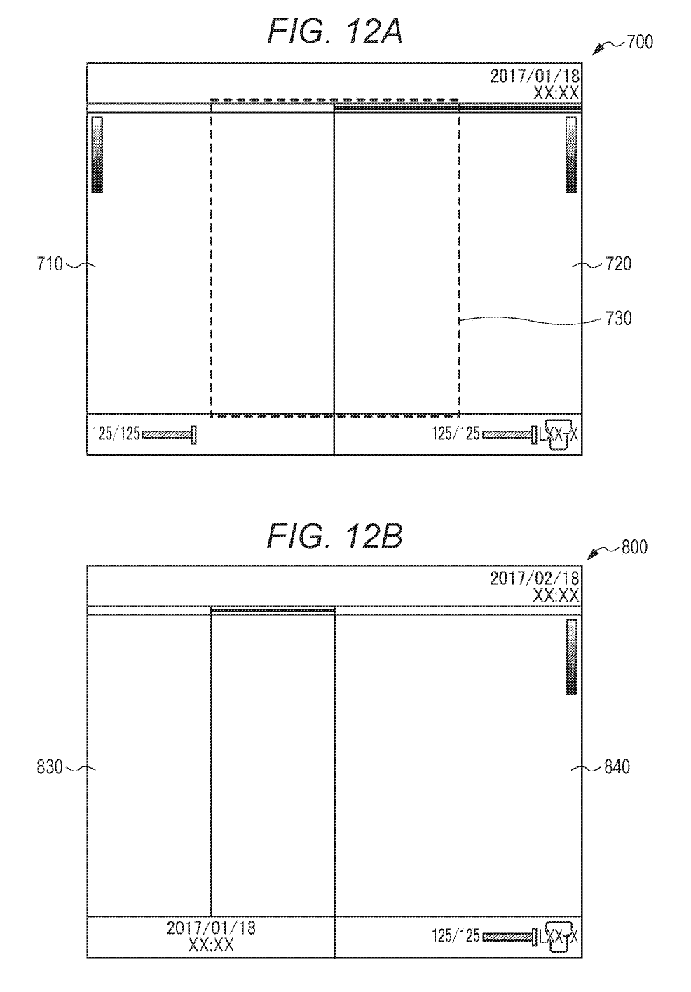

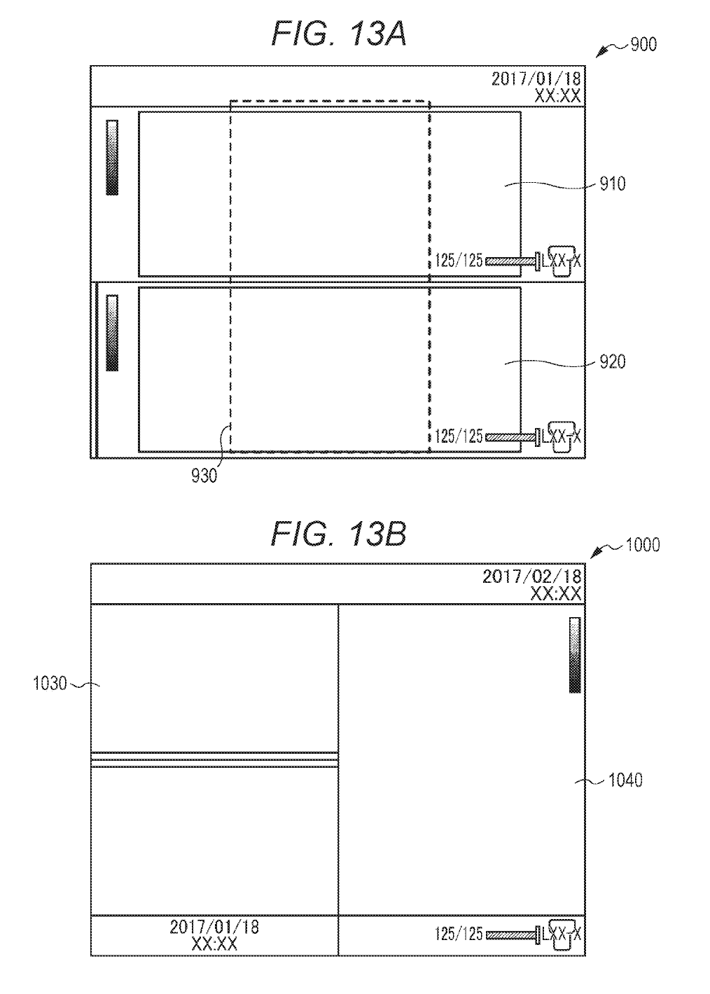

A third modification of the above embodiment will be described with reference to FIG. 11. FIG. 11 is a flowchart illustrating third multiple-image comparison display processing.

In the present modification, the ultrasound diagnosis apparatus 1 is used as an apparatus, similarly to the above-described embodiment. It is assumed, however, that the ROM of the controller 108 stores the second multiple-image comparison display program instead of the third multiple-image comparison display program. In addition, it is assumed that the setting information set and stored beforehand in the storage 109 includes the comparison image display position information alone.

Next, operation of the ultrasound diagnosis apparatus 1 according to the present modification will be described. The third multiple-image comparison display processing will be described with reference to FIG. 11. The third multiple-image comparison display processing is processing of using graphic information detection information and the setting information to perform comparison display of an appropriate ultrasound image contained in the two-image-saved image data stored in the storage 109 and a current frozen image, being arranged in parallel.

As illustrated in FIG. 11, step S81 is similar to step S31 of the first multiple-image comparison display processing in FIG. 6. Subsequently, the controller 108 detects active screen lines, grayscale bars, cine bars as graphic information together with their position information from the image of the two-image-saved image data read in step S81 by image analysis, and determines the detected position information of the graphic information as detection information (step S82). Exemplary image analysis is template matching of each of the pieces of graphic information.

Step S82 generates, for example, position information indicating that the active screen line 330 is arranged on the upper side (upper right side of the screen) of the ultrasound image 320, position information indicating that each of the grayscale bars 311 and 321 is respectively arranged on the left-right side of each of the ultrasound images 310 and 320, and position information indicating that each of the cine bars 312 and 322 is respectively arranged on the lower side (lower side of the screen) of each of the ultrasound images 310 and 320, in the two-image-saved image 300 of FIG. 4. As another example, step S82 generates position information indicating that the active screen line 430 is arranged on the left side (lower left side of the screen) of the ultrasound image 420, position information indicating that each of the grayscale bars 411 and 421 is respectively arranged on the left side (left side of the screen) of each of the ultrasound images 410 and 420, and position information indicating that each of the cine bars 412 and 422 is respectively arranged on the lower right side (lower right side of the screen) of each of the ultrasound images 410 and 420, in the two-image-saved image 400 of FIG. 5.

Subsequently, the controller 108 determines (step S83) the image arrangement format and an ultrasound image as a comparison image among the two-image-saved image data on the basis of the detection information obtained in step S82.

In a case where the two-image-saved image 300 is read in step S81, for example, position information indicating that the active screen line 330 is arranged on the upper side (upper right side of the screen) of the ultrasound image 320, position information indicating that each of the grayscale bars 311 and 321 is respectively arranged on the left-right side (left-right side on the screen) of each of the ultrasound images 310 and 320, and position information indicating that each of the cine bars 312 and 322 is respectively arranged on the lower side (lower side of the screen) of each of the ultrasound images 310 and 320, among the detection information, is a feature of a case where the image arrangement format is the left-right format. Therefore, in step S85, the left-right format is determined as the image arrangement format. Similarly, the position information indicating that the active screen line 330 is arranged on the upper side (upper right side of the screen) of the ultrasound image 320 among the detection information indicates that it is preferable that the ultrasound image 320 be designated as the comparison image when it is in the active state. Accordingly, the ultrasound image 320 on the right side among the two-image-saved image data is determined as the comparison image in step S85

Moreover, in a case where the two-image-saved image 400 is read in step S81, for example, position information indicating that the active screen line 430 is arranged on the left side (left side of the screen) of the ultrasound image 420, position information indicating that each of the grayscale bars 411 and 421 is respectively arranged on the left side (left side of the screen) of each of the ultrasound images 410 and 420, and position information indicating that each of the cine bars 412 and 422 is respectively arranged on the lower right side (lower right side of the screen) of each of the ultrasound images 410 and 420, among the detection information, is a feature of a case where the image arrangement format is the upper-lower format. Accordingly, in step S83, the upper-lower format is determined as the image arrangement format. Moreover, the position information indicating that the active screen line 430 is arranged on the left side (lower left side of the screen) of the ultrasound image 420 among the detection information indicates that it is preferable that the ultrasound image 420 be designated as the comparison image when it is in the active state. Accordingly, the ultrasound image 420 on the right side among the two-image-saved image data is determined as the comparison image in step S83.

Step S84 is similar to step S64 of the second multiple-image comparison display processing in FIG. 10. Steps S85 to S90 are respectively similar to steps S34 to S39 of the first multiple-image comparison display processing in FIG. 6. Note that the graphic information used in detecting the arrangement (position information) is not limited to the active screen line, the grayscale bar, or the cine bar. It is allowable to use a configuration to detect arrangement of other graphic information (position information) such as a mark indicating the type of the ultrasound probe 1b.

According to the present modification as described above, the controller 108 performs image analysis on the obtained two-image-saved image data to detect the position information of the active screen line, the grayscale bar, and the cine bar, as graphic information, and obtains the image arrangement format corresponding to the detected position information. This makes it possible to appropriately perform comparison display of the past comparison image of the two-image-saved image data and the current frozen image in the image arrangement format of the two-image-saved image data.

In addition, the controller 108 performs image analysis on the obtained two-image-saved image data to detect the position information of the active screen line as graphic information indicating the active state of the ultrasound image, and obtains the comparison image designation information corresponding to the detected position information. This makes it possible to perform comparison display of an appropriate past comparison image that is in the active state at the time of saving among the two-image-saved image data, and the current frozen image.

The description in the above embodiment is an example of a preferred ultrasound diagnosis apparatus according to the present invention, and the present invention is not limited thereto. For example, at least two of the above-described embodiments, modifications, and the following configurations may be appropriately combined with each other. More specifically, it is allowable to use a configuration for the multiple-image comparison display processing, in which the two-image comparison screen is displayed using the image arrangement format based on the supplementary information and the comparison image designation information based on the position information of the graphic information.

While the above-described embodiment and the modification use a configuration in which the two-image-saved image data is generated from the two ultrasound images (current frozen images) generated by transmitting and receiving ultrasound waves to and from the subject, the present invention is not limited to this configuration. It is allowable to use a configuration, for example, of generating two-image-saved image data from one ultrasound image generated and stored in the past and from one ultrasound image (current frozen image) generated by transmitting and receiving ultrasound waves to and from the subject. Furthermore, it is allowable to use a configuration in which two-image-saved image data corresponding to the two-image comparison screen displayed in the first to third multiple-image comparison display processing is generated and stored in the storage 109.

Moreover, while the above-described embodiment and the modifications use a configuration of using the two-image-saved image data as the multiple-image-saved image data in which two ultrasound images are arranged in parallel in the left-right or the upper-lower format, for the comparison display, the present invention is not limited to this configuration. It is allowable to use a configuration of using a multiple-image-saved image data in which three or more ultrasound images are arranged in one screen, for the comparison display. For example, it is allowable to use a configuration to first determine a comparison image on the basis of the comparison image designation information from among multiple-image-saved image data containing three or more ultrasound images, and subsequently perform comparison display of the comparison image and the current frozen image being arranged in a single screen.

Moreover, while the above-described embodiment and the modifications use a configuration in which the comparison display is performed between the comparison image (still image) of the multiple-image-saved image data as a still image and the current frozen image as the current image (still image), the present invention is not limited to this configuration. For example, it is allowable to use a configuration in which the comparison display is performed between a comparison image (moving image) of multiple-image-saved image data as a moving image, and a live image as a current image (moving image). Furthermore, it is also allowable to use a configuration in which the comparison display is performed between the comparison image (still image) of the multiple-image-saved image data as a still image and the live image, or in which the comparison display is performed between the comparison image (moving image) of the multiple-image-saved image data as a moving image and the current frozen image as a still image.

Moreover, while the above-described embodiment and the modifications use a configuration in which the controller 108 reads and obtains the multiple-image-saved image data (and supplementary information) from the storage 109, the present invention is not limited to this configuration. For example, it is allowable to use a configuration in which the ultrasound diagnosis apparatus 1 includes a communication part to communicate with an external apparatus, the multiple-image-saved image data (and supplementary information) is stored in an external apparatus such as an external image server beforehand, and the controller 108 obtains the multiple-image-saved image data (and supplementary information) via the communication part by sending a request to and receiving the data from the external apparatus.

In addition, detailed configurations and detailed operation of each of the apparatuses configuring the ultrasound diagnosis apparatus 1 according to the present embodiments can be appropriately modified without departing from the spirit and scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasound diagnosis apparatus comprising:
a transmitter that outputs a drive signal to an ultrasound probe that transmits and receives ultrasound waves;
a receiver that obtains a reception signal from the ultrasound probe;
an image generator that generates ultrasound image data from the reception signal;
a multiple-image-saved image data acquisition part that obtains multiple-image-saved image data containing a plurality of ultrasound images generated in the past within a single screen;
an information acquisition part that obtains an image arrangement format of the plurality of ultrasound images of the obtained multiple-image-saved image data; and
a display controller that performs comparison display of a comparison image among the plurality of ultrasound images of the multiple-image-saved image data and a current image based on the generated current ultrasound image data onto a single screen of the display part in accordance with the obtained image arrangement format.

2. The ultrasound diagnosis apparatus according to claim 1,
wherein the information acquisition part obtains the image arrangement format from supplementary information associated with the obtained multiple-image-saved image data, the supplementary information containing the image arrangement format of the multi-image-saved image data.

3. The ultrasound diagnosis apparatus according to claim 1,
wherein the information acquisition part performs image analysis on the obtained multiple-image-saved image data to detect position information of graphic information, and obtains the image arrangement format corresponding to the position information.

4. The ultrasound diagnosis apparatus according to claim 1,
wherein the information acquisition part obtains comparison image designation information for designating a comparison image from among the plurality of ultrasound images of the obtained multiple-image-saved image data, and
the display controller performs comparison display of the comparison image among the plurality of ultrasound images of the multiple-image-saved image data and a current image based on the generated current ultrasound image data onto a single screen of the display part in accordance with the obtained image arrangement format and the obtained comparison image designation information.

5. The ultrasound diagnosis apparatus according to claim 2,
wherein the information acquisition part obtains comparison image designation information for designating a comparison image from among the plurality of ultrasound images of the obtained multiple-image-saved image data, and
the display controller performs comparison display of the comparison image among the plurality of ultrasound images of the multiple-image-saved image data and a current image based on the generated current ultrasound image data onto a single screen of the display part in accordance with the obtained image arrangement format and the obtained comparison image designation information.

6. The ultrasound diagnosis apparatus according to claim 3,
wherein the information acquisition part obtains comparison image designation information for designating a comparison image from among the plurality of ultrasound images of the obtained multiple-image-saved image data, and
the display controller performs comparison display of the comparison image among the plurality of ultrasound images of the multiple-image-saved image data and a current image based on the generated current ultrasound image data onto a single screen of the display part in accordance with the obtained image arrangement format and the obtained comparison image designation information.

7. An ultrasound diagnosis apparatus comprising:
a transmitter that outputs a drive signal to an ultrasound probe that transmits and receives ultrasound waves;
a receiver that obtains a reception signal from the ultrasound probe;
an image generator that generates ultrasound image data from the reception signal;
a multiple-image-saved image data acquisition part that obtains multiple-image-saved image data containing a plurality of ultrasound images generated in the past within a single screen;
an information acquisition part that obtains comparison image designation information to designate a comparison image from among the plurality of ultrasound images of the obtained multiple-image-saved image data; and
a display controller that performs comparison display of a comparison image among the plurality of ultrasound images of the multiple-image-saved image data and a current image based on the generated current ultrasound image data onto a single screen of the display part in accordance with the obtained comparison image designation information.

8. The ultrasound diagnosis apparatus according to claim 7,
wherein the information acquisition part obtains the comparison image designation information from supplementary information associated with the obtained multiple-image-saved image data, the supplementary information containing the comparison image designation information designating an ultrasound image that is in an active state at the time of saving of the multiple-image-saved image data, as the comparison image.

9. The ultrasound diagnosis apparatus according to claim 4,
wherein the information acquisition part obtains the comparison image designation information from supplementary information associated with the obtained multiple-image-saved image data, the supplementary information containing the comparison image designation information designating an ultrasound image that is in an active state at the time of saving of the multiple-image-saved image data, as the comparison image.

10. The ultrasound diagnosis apparatus according to claim 7,
wherein the information acquisition part performs image analysis on the obtained multiple-image-saved image data to detect position information of graphic information indicating an active state of the ultrasound image, and obtains comparison image designation information corresponding to the detected position information.

11. The ultrasound diagnosis apparatus according to claim 4,
wherein the information acquisition part performs image analysis on the obtained multiple-image-saved image data to detect position information of graphic information indicating an active state of the ultrasound image, and obtains comparison image designation information corresponding to the detected position information.

12. The ultrasound diagnosis apparatus according to claim 7,
wherein the information acquisition part obtains the comparison image designation information from setting information containing the comparison image designation information fixedly set beforehand.

13. The ultrasound diagnosis apparatus according to claim 4,
wherein the information acquisition part obtains the comparison image designation information from setting information containing the comparison image designation information fixedly set beforehand.

14. The ultrasound diagnosis apparatus according to claim 7,
wherein the information acquisition part obtains the comparison image designation information from setting information containing comparison image designation information set beforehand in accordance with an operation input.

15. The ultrasound diagnosis apparatus according to claim 4,
wherein the information acquisition part obtains the comparison image designation information from setting information containing comparison image designation information set beforehand in accordance with an operation input.
